(19) United States
(12) Plant Patent Application Publication
(10) Pub. No.: US 2007/0261144 P1
(43) Pub. Date: Nov. 8, 2007

(54) Variety of canna named 'LON01'

(76) Inventor: Geoff Jewell, Otaki (NZ)

Correspondence Address: FULWIDER PATTON LLP, 200 OCEANGATE, SUITE 1550, LONG BEACH, CA, 90802, US

(21) Appl. No.: 11/416,520

(22) Filed: May 3, 2006

(51) Int. Cl. A01H 5/00

(52) U.S. Cl. PLT/313

(57) ABSTRACT

A new and distinct cultivar of Canna plant named ‘LON01’ characterized by browned burgundy leaves tending to olive green as the leaf ages.

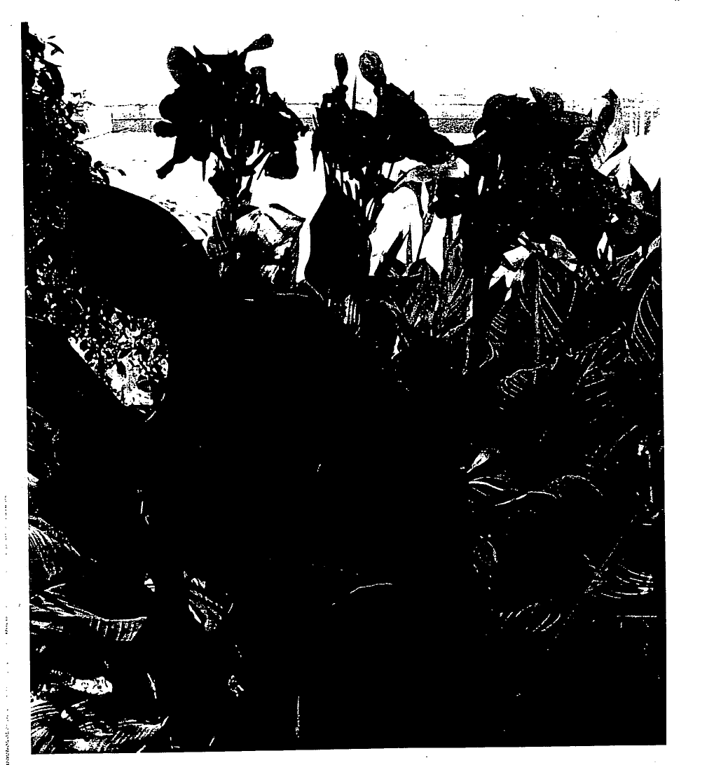

### BACKGROUND OF THE NEW PLANT

The present invention comprises a new and distinct cultivar of Canna, botanically known as Canna hybrida and further known by the varietal name ‘LON01’ and the trade name ‘Tropicanna Black.’ The new variety was discovered through a selective breeding program by Greg Goff at Lone Star International, S.A. de C.V., Av. Union 274-A, Col. Americana, Guadalajara, Jalisco Mexico 44100. The seedlings were selected on Aug. 21, 2001, and the new variety is a product of open pollination. The new cultivar was selected from the progeny by the inventor Greg Goff at Lone Star International, S.A. de C.V., Av. Union 274-A, Col. Americana, Guadalajara, Jalisco Mexico 44100.

### SUMMARY OF THE INVENTION

The new variety is the result of a selective breeding program taken from selected seedlings in the Nursery at Lone Star International, S.A. de C.V. in the year 2001. These seedlings were selected and propagated through rhizome divisions selecting for differences in color variation of the interveinal striping.

The new cultivar has since been asexually reproduced by rhizome divisions at Anthony Tesslaar International, Pty Ltd., Silvan, Victoria, Australia and has been repeatedly asexually reproduced by Anthony Tesslaar International Pty Ltd. Continued observations from the vegetative divisions have confirmed that the distinguishing features of this new cultivar came true, remain stable and are retained through successive propagation.

A comparative trial of the new cultivar with its closest known variety ‘Phasion’, trade name “Tropicanna” was conducted at Clyde, VIC, Australia in February 2006, in a garden environment.

### BRIEF DESCRIPTION OF THE DRAWINGS

The accompanying photograph illustrates the new cultivar, the color being as nearly true as possible with color photographs of this type.

### DETAINED BOTANICAL DESCRIPTION OF THE NEW PLANT

The following detailed description sets forth characteristics of the new cultivar. The data which defines each characteristic was collected from the comparative trial identified above, using R.H.S. color cards issued in 1995.

### THE PLANT

| | | |
|---|---|---|
| Classifi- | Botanical- | Canna hybrida |
| cation: | Commercial- | ‘LON01’ |
| | Origin- | Selected seedling |
| | Propagation- | Rhizome division |
| Form: | Type- | Dense, upright mass of stems topped by |
| | | flowering panicles, slowly expanding |
| | | in width by rhizomes beneath the soil |
| | | surface |
| | Height- | Up to 1500 mm |
| | Width- | Up to 500 mm |
| | Growth Rate | Flowers in first season from Summer to |
| | and Habit- | well into Autumn. Flower truss lasts |
| | | approximately two weeks, each individual |
| | | floret lasting two to three days |
| | Strength- | free standing |
| | Stem- | 20-30 mm |
| Leaves: | Arranged- | spirally |
| | Shape- | Lamina is large, broad, ovate and |
| | | elliptic, with prominent venation with |
| | | deep veins and long midrib |
| | Length- | Mean 560 mm |
| | Width- | Mean 233 mm |
| | Margin- | Entire |
| | Texture- | Thick, smooth but with prominent rib |
| | | structure, and somewhat waxy feel |
| | Color- | Browned Burgundy tending to olive green |
| | | as leafages. Closest RHS 200A-187A (PMS |
| | | 5185 C), ageing to 147A (primary color) |

### INFLORESCENCE

| General: | | |
|---|---|---|
| Bome: | | |
| Bud: | Shape- | Lanceolate - gladiolus like |
| | | (elongated spikes) |
| Sepal: | Quantity - | 3 |
| | Length- | |
| | Width- | |
| | Form- | Resembles petal although |
| | | completely rolled |
| Petals: | Time of flowering is first season from Summer | |
| | to well into Autumn. The flower truss lasts | |
| | approximately 2 weeks, each individual floret | |
| | lasts 2-3 days. | |
| | Quantity- | 5 |
| | Size- | Medium |
| | Arrangement- | Overlapping |
| | Length- | 75-90 mm |
| | Width- | 50-60 mm |
| | Color- | Midzone outside - |
| | | Red Orange Group RHS42B |
| | | Midzone inside - |
| | | Red Orange Group RHS33B |
| | | Margin inside - |
| | | Red Orange Group RHS33B |
| Flowers: | Quantity- | 15-30 |
| | Size- | Floret medium |
| | Profile Upper- | Flattened Concave |
| | Profile Lower- | “V” shaped |
| | Color- | |
| Floret: | Length- | Approximately 80 mm |
| | | (fully open floret) |
| Pedicel | Length | Peduncle spike |
| | | approximately 300 mm |
| Fragrance: | None detected | |
| Persistence: | Disease Resistance- | Very tolerant |
| | Lasting Quality- | Regenerating |
| Time of | early, med, late | Medium |
| Flowering | | |
| Length of | med, long | Medium |
| Flowering | | |
| period | | |
| Color midzone | | 42B RHS (PMS 179 C) |
| outside | | |
| Color midzone | | CA. 33B (PMS 172 C) |
| inside | | |
| Color margin | | 33B |
| inside | | |
| Color | | yellowish white |
| Basal Spot | | |

### REPRODUCTIVE ORGANS

| | | |
|---|---|---|
| Stamens: | | |
| Filaments: | Color- | light yellow to white |
| Anther: | Orange | |
| Pistils: | Style- | Cream |
| | Shape | Flattened lobed shaped blade of orange-red, |
| | | length 70 mm |
| Seed Vessel | small, med, | Medium, tri-lobed, spiky anemone like |
| Size: | large | |
| | Color- | Reddish purple with some green |
| Seed | | small, ball-shaped, attached to center pier, |
| | | approximately 8 to vessel |

### GENERAL CHARACTERISTICS

The varietal is characterized by large, broad leaves having undulations of margin that are strong in juvenile becoming weak with age. Reflexion of margin is absent, and venation is deeply indented. The predominant color is browned burgundy (RHS 187A) becoming white in center, lateral venations are raised. The aspect is upright, as the leaves unfurl as they grow from the stalk. As the leaf ages, the browned burgundy tends to olive green (147A), with the color consistent on the lower and upper sides. For each growth spike, one to three umbels are present flowering first at the apex, second below first peduncle, and third from a lower leaf axil. The varietal flowers in the first season from Summer to well into Autumn, where each flower lasts approximately two weeks and each floret lasts 2-3 days.

I claim:

1. A new and distinct variety of Canna plant, substantially as shown and described.
